(19) United States
(12) Patent Application Publication
(10) Pub. No.: US 2006/0141523 A1
(43) Pub. Date: Jun. 29, 2006

(54) Integrated method for PCR cleanup and oligonucleotide removal

(75) Inventors: Lawrence Greenfield, Eugene, OR (US); Douglas A. Bost, San Mateo, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/357,822

(22) Filed: Feb. 17, 2006

Related U.S. Application Data
(62) Division of application No. 10/202,611, filed on Jul. 23, 2002.

(51) Int. Cl. C12Q 1/68; C12P 19/34

(52) U.S. Cl. 435/6; 435/91.2

(57) ABSTRACT

A method is provided for purifying a desired polynucleotide product by removing unincorporated oligonucleotides from a polymerase or ligase reaction mixture.

### CROSS REFERENCE TO RELATED APPLICATIONS

This application is a Divisional Application of U.S. Non-provisional application Ser. No. 10/202,811, filed Jul. 23, 2002, the contents of which are incorporated by reference herein.

### BACKGROUND

Nucleic acid sequence analysis is extremely important in many research, medical, and industrial fields. See, e.g., Caskey, Science 236:1223-1228 (1987); Landegren et al, Science 242:229-237 (1988); and Arnheim et al, Ann. Rev. Biochem. 61:131-156 (1992). The most commonly used sequence analysis technique is polymerase chain reaction (PCR). PCR and other sequence determination techniques involve extension of an oligonucleotide primer with a polymerase. Extension of a primer with a polymerase also occurs in vivo in DNA replication and in transcription of DNA to form RNA.

Fidelity of DNA replication in vivo is maintained, in part, by a 3′-to-5′ exonuclease proof-reading activity of the DNA polymerase. When an incorrect nucleotide is incorporated and forms a mismatch with the template, it is removed by the 3′-to-5′ exonuclease. The thermostable DNA polymerase most widely used for PCR, however, Thermus aquaticus (Taq) polymerase, lacks a 3′-to-5′ exonuclease.

Other methods of sequence determination or nucleic acid analysis involve ligation of oligonucleotides, or involve both ligation of oligonucleotides and polymerase extension of oligonucleotides. One technique is the oligonucleotide ligation assay (OLA) of Whiteley et al., U.S. Pat. No. 4,883,750. The method is used to determine the presence or absence of a target sequence in a sample of denatured template nucleic acid. Two oligonucleotide probes are designed so they will hybridize to the target sequence with the 5′ base of one oligonucleotide abutting the 3′ base of the other. If these two bases form perfect hybrds with the target sequence of the template DNA, then the oligonucleotides can be ligated together by DNA ligase. If the template DNA contains a mutation at one of those two bases in the target sequence, the oligonucleotides cannot be ligated. If a thermostable ligase is used, the reaction can be carried out for multiple cycles, just as in PCR. This can greatly improve the signal to noise ratio. (See Wu and Wallace, Genomics 4:560 (1989); Barany, Proc. Natl. Acad. Sci. USA 88:189(1991).) Assays that combine OLA and PCR are described in Eggerding, U.S. Pat. No. 6,130,073; and Nickerson et al., Proc. Natl. Acad. Sci. USA 87:8923-8927 (1990).

In PCR and other polymerase-based assays using oligonucleotides, as well as in ligation-based assays, unextended or unligated oligonucleotides often need to be removed from the reaction mixture for subsequent analysis steps. This is true, for instance, in nested PCR and sequencing of PCR products, or when the amplified product is to be hybridized to a sequence to which the primer would competitively hybridize. Hence, there is a need for techniques that quickly and easily remove unextended oligonucleotides from polymerase and ligase reaction mixtures.

### SUMMARY OF THE INVENTION

One embodiment of the present invention provides a method for removing unincorporated oligonucleotides from a reaction mixture. The method involves the following steps: (a) forming a mixture containing a DNA polymerase or nucleic acid ligase, a nuclease, an upstream oligonucleotide having a 3′ portion and a 5′ portion (wherein the 3′ portion has a 3′ recognition group and a 3′ terminal nucleotide), and a template nucleic acid, (b) digesting the 3′ portion of the upstream oligonucleotide with the nuclease, (c) extending the digested upstream oligonucleotide with the polymerase or ligating the digested upstream oligonucleotide to a downstream oligonucleotide with the ligase, wherein the extending or ligating forms a polynucleotide product, and (d) contacting the mixture with a substrate having binding groups that bind the 3′ recognition group, to remove unincorporated upstream oligonucleotides from the reaction mixture. The DNA polymerase or nucleic acid ligase and the nuclease used in the method may be the same or separate enzyme complexes.

In this embodiment, the recognition group generally is attached to the 3′ terminal nucleotide of the upstream oligonucleotide. The recognition group may prevent the upstream oligonucleotide from being extended or ligated until the 3′ recognition group is removed. The 3′ portion of the upstream oligonucleotide may be non-complementary with the template, so that the 3′ portion, along with the 3′ recognition group, is more likely to be removed by a 3′-to-5′ proofreading exonuclease.

Another embodiment of the present invention provides a method for removing unincorporated oligonucleotides from a reaction mixture. The method involves the following steps: (a) forming a mixture containing a nucleic acid ligase, a nuclease, a downstream oligonucleotide having a 3′ portion and a 5′ portion (wherein the 5′ portion comprises a 5′ recognition group and a 5′ terminal nucleotide), and a template nucleic acid, (b) digesting the 5′ portion of the downstream oligonucleotide with the nuclease, (c) ligating the digested downstream oligonucleotide to an upstream oligonucleotide with the ligase, wherein the ligating forms a polynucleotide product, and (d) contacting the mixture with a substrate having binding groups that bind the 5′ recognition group to remove unincorporated downstream oligonucleotides from the reaction mixture. The nucleic acid ligase and nuclease may be the same or separate enzyme complexes.

In this embodiment, the 5′ recognition group generally is attached to the 5′ terminal nucleotide of the downstream oligonucleotide, and prevents the downstream oligonucleotide from being ligated until the 5′ reognition group is removed.

### DETAILED DESCRIPTION OF THE INVENTION

Definitions

“Nucleic acid polymerase” is an enzyme that catalyzes the formation of a nucleic acid product from nucleoside triphosphates, using either a DNA or RNA template. Nucleic acid polymerases include both RNA polymerases and DNA polymerases.

“DNA polymerase” means a polymerase that synthesizes DNA. This includes both DNA-directed DNA polymerases (using DNA as a template) and RNA-directed DNA polymerases or reverse transcriptases (using RNA as a template).

“Oligonucleotide” refers to a polynucleic acid or a series of covalently-linked nucleic acid bases that are capable of hybridizing to a second nucleic acid sequence. When hybridized to a template under appropriate conditions, an oligonucleotide can serve as a substrate which is extended by a DNA polymerase adding nucleotides to it. The oligonucleotide can also serve as a substrate for a ligase. When an upstream oligonucleotide hybridizes to a template adjacent to a downstream oligonucleotide, the two oligonucleotides can be ligated. The oligonucleotides can consist of predominantly deoxyribonucleotides or ribonucleotides, or a mixture of both. The oligonucleotides can also contain modified nucleotides. Usually monomers are linked by phosphodiester bonds to form polynucleotides. However, the nucleoside monomers of the oligonucleotides can be linked by other linkages. Oligonucleotides can be any length sufficient to specifically hybridize to the target template and be extended by a polymerase or ligated by a ligase after digestion with the nuclease. This can range from as few as six nucleotides to over a thousand. Typically the oligonucleotides will be from about 9 nucleotides in length to about 100 nucleotides, about 10 nucleotides to about 50, or about 10 to about 25 nucleotides. After cleavage by the nucleases to remove the 3′ portion of the oligonucleotide containing the 3′ recognition group (or to remove the 5′ portion of the oligonucleotide containing the 5′ recognition group when the recognition group exists in the 5′ portion for some ligation reactions), the oligonucleotide contains a sufficient number of hybridizing nucleotides to hybridize to the template stably enough to permit extension by the polymerase or ligation by the ligase. The sequence of nucleotide monomers in the oligonucleotides may be interrupted or appended by other groups, such as recognition groups. The term “oligonucleotide” also encompasses analogs of naturally occurring polynucleotides. Examples of such analogs include, but are not limited to, peptide nucleic acid and LOCKED NUCLEIC ACID (LNA). For disclosures of peptide nucleic acid, see, e.g., Egholm et al., Science 254:1497 (1991); WO92/20702; and U.S. Pat. Nos. 6,180,767 and 5,714,331. Peptide nucleic acid has a peptide backbone, instead of a sugar-phosphate backbone, to which the bases are connected.

“Modified nucleotides” include, for example, dideoxyribonucletides and synthetic nucleotides having modified base moieties or modified sugar moieties, e.g., as described in Scheit, Nucleotide Analogs (John Wiley, New York 1980) and Uhlman and Peyman, Chemical Reviews 90:543-584 (1990). Such analogs include synthetic nucleotides designed to enhance binding properties, reduce degeneracy, and increase specificity. The term “modified nucleotides” also includes nucleotides blocked at their 3′ terminus to prevent extension or ligation, such as 3′-dideoxyribonucleotides, 3′-deoxyribonucleotides, 3′-NH₂, 3′-SH, 3′-phosphoglycoaldehyde, and 3′-Pᵢ nucleotides, and nucleotides to whose 3′-hydroxyls a recognition group such as biotin has been attached. The term “modified nucleotides” also includes nucleotides blocked at their 5′ terminus to prevent ligation of the 5′ terminus, such as 5′-deoxyribonucleotides, 5′-NH₂, 5′-SH, and nucleotides to whose 5′-hydroxyls a recognition group such as biotin has been attached. The term “modified nucleotides” also includes nucleotides to which a recognition group has been attached at a position other than the 3′- or 5′-hydroxyl. The term “modified nucleotide” also includes normal ribonucleotides in the context of an oligonucleotide whose hybridizing portion is predominantly DNA. The term “modified nucleotide” also includes nucleotides lacking a base, referred to herein as “AP nucleotides.” The AP stands for apyrimidinic or apurinic, depending on whether the missing base is a pyrimidine or purine, respectively.

As used herein, “nucleotide” includes moieties consisting essentially of a base, sugar, and phosphate or polyphosphate, as well as a moiety in which the base, sugar, or phosphate is modified. It includes also moieties in which the phosphate is absent or replaced by a chemically different group. For instance, “polynucleotide” as used herein includes polymers in which the nucleosides or modified nucleosides are linked by modified phosphodiester linkages, such as methyl phosphonate linkages or phosphorothionate linkages. The term “nucleotide” also includes AP nucleotides, which lack a base, and moieties in which a non-basic group, such as glycerol, replaces the base.

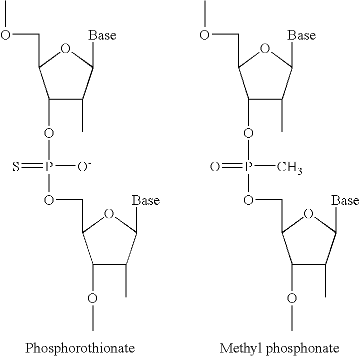

“Template nucleic acid” includes both RNAs and DNAs. It refers to the polynucleic acid to which the oligonucleotides bind and which serves as template for extension of the oligonucleotide by the polymerase or ligation of the oligonucleotides by a ligase.

“Recognition group” refers to a chemical group attached to the oligonucleotide that can be recognized and bound specifically by the binding group. The recognition group can be covalently attached or non-covalently attached. Preferably it is covalently attached. If it is non-covalently attached, the attachment is preferably substantially stable under the conditions of the polymerase or ligase reaction and of the contacting with the binding groups. The recognition group can be attached at any synthetically feasible position on any nucleotide or nucleoside residue of the oligonucleotide. For instance, the recognition group can be attached at the 3′ hydroxyl of the 3′-terminal nucleotide or 5′ hydroxyl of the 5′-terminal nucleotide. The recognition group can also be attached to an internal residue of the oligonucleotide. When the recognition group has two appropriate positions for attachment, the recognition group can form part of the polynucleic acid backbone, being flanked on both sides by nucleotides or nucleosides.

As used herein, “3′ terminal nucleotide” refers to the nucleotide that is the furthest in the 3′ direction in the oligonucleotide. This nucleotide may have a free 3′-OH or may have its 3′ hydroxyl attached to a blocking group or to the 3′ recognition group, or absent as in a 3′ deoxynucleotide. The oligonucleotide may also be circularized, so that the 3′ terminal nucleotide is attached, such as through its 3′ hydroxyl, to another nucleotide of the oligonucleotide.

As used herein, “5′ terminal nucleotide” refers to the nucleotide that is the furthest in the 5′ direction in the oligonucleotide. This nucleotide may have a free 5′-OH or 5′ mono-, di-, or tri-phosphate, or may have its 5′ hydroxyl attached to a blocking group or to the 5′ recognition group, or absent as in a 5′ deoxynucleotide. The oligonucleotide may also be circularized, so that the 5′ terminal nucleotide is attached, such as through its 5′ phosphate, to another nucleotide of the oligonucleotide.

“Size exclusion chromatography resin” refers to a solid matrix of any type, whether made of natural or synthetic materials, suitable for use in size exclusion chromatography. This includes, for instance, dextran, agarose, polyacrylamide, and mixtures thereof.

“Binding group” refers to a chemical group that will specifically bind with the recognition group under the conditions of the step of contacting the reaction mixture with the substrate comprising the binding group. The binding can be by covalent or non-covalent interactions. The non-covalent interactions can be, for instance, ionic or hydrophobic, or a mixture thereof. The interactions should be strong enough that most, or even substantially all, of the oligonucleotide containing the recognition group is bound to the substrate containing the binding group and therefore is removed from the reaction mixture. “Substantially all” in this context means at least 80%. In alternative embodiments, at least 90%, at least 95%, or at least 99% of the oligonucleotide containing the recognition group is bound to substrate containing the binding group and therefore is removed from the reaction mixture. The binding group can be, for instance, an antibody, protein, carbohydrate, metal cation, or other chemical group.

Suitable recognition groups include digoxygenin, fluorescein, and biotin. Suitable binding groups include an anti-digoxygenin antibody to bind digoxygenin; an anti-fluorescein antibody to bind fluorescein; and an anti-biotin antibody, streptavidin, or avidin to bind biotin. Another suitable recognition group is polyhistidine. In this case, a suitable binding group is the Ni²⁺ cation. Typically, the Ni cation will be ligated with a chelator. The polyhistidine can contain almost any length of consecutive histidine residues, provided the peptide interacts stably with Ni cations. Typically approximately a 6-mer of histidine will be used. Another suitable recognition group-binding group pair is a recognition group that comprises phenylboronic acid (PBA) and a binding group that comprises salicylhydroxamic acid (SHA), or vice versa. Groups containing PBA (on the left) and SHA (on the right) are shown below, along with their reaction to form a PBA-SHA complex. R indicates the point of attachment to the oligonucleotide or the binding group support. The point of attachment can be at any chemically feasible position, not just those shown. The term “group comprising phenylboronic acid” also includes other groups that retain the PBA functionality, such as groups in which the phenyl group is substituted, e.g., by a second boronic acid group. Likewise, the term “group comprising salicylhydroxamic acid” includes other groups that retain the SHA functionality, e.g., those in which the phenyl ring is substituted, provided that the PBA- and SHA-comprising groups retain their ability to bind one another. See the products of Prolinx Inc., Bothell, Wash.

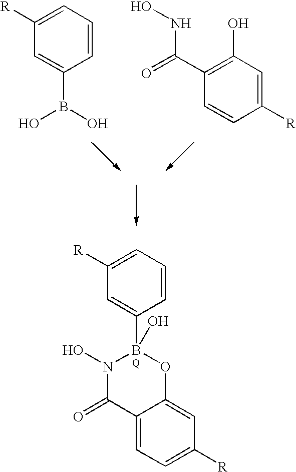

The PBA-SHA linkage is reversible upon addition of a competitor such as phenylboronic acid or phenyl-1,3-diboronic acid. See, e.g., U.S. Pat. Nos. 5,594,111; 6,156,884; and 5,623,055; and product instructions from Prolinx, Inc., Redmond, Wash.

“RNAse H” as used herein means an enzyme that cleaves RNA that is part of a RNA:DNA heteroduplex. Incorporation of one or more RNA residues in an oligonucleotide allows the oligonucleotide to be cleaved at the hybridized RNA residues when the oligonucleotide is hybridized to a DNA template strand. Some RNAse Hs require only one ribonucleotide in an oligonucleotide as substrate. Others require a segment of up to four ribonucleotides. RNAse H activity can be found in some polymerases, including reverse transcriptase. RNAse H can also be a separate enzyme. One suitable RNAse H is Thermus thermophilus, or Tth, RNAse H. Other suitable RNAse H enzymes include human and E. coli RNAse Hs.

As used herein, “enzyme complex” refers to a protein. The protein may have one or more polypeptide chains. If it has more than one polypeptide chain, the polypeptides are normally associated together. An enzyme complex can have one enzyme activity or more than one enzyme activity. For instance, a single enzyme complex may have both polymerase and nuclease activities, or it may have both ligase and nuclease activities. The active sites for the more than one enzyme activities can be overlapping or the same active site, or they can be spatially separated on the enzyme complex.

“5′ kinase” refers to a kinase that attaches a phosphate to a 5′-OH of a nucleic acid.

“3′ phosphatase” refers to an enzyme that removes a phosphate from a 3′-phosphonucleotide to yield a free 3′-OH group.

“AP endonuclease” refers to any enzyme that cleaves at the 5′ side of an AP nucleotide (a nucleotide that lacks a base), yielding a free 3′-OH on the adjacent nucleotide and a 5′-phosphate on the AP nucleotide.

As used herein, “upstream” means in the direction of the oligonucleotide's 5′ end, and “downstream” means in the direction of the oligonucleotide's 3′ end. When two oligonuceotides hybridze to a template, the oligonucleotide that is in the most 5′ position, i.e., hybridized to the most 3′ position of the template, is referred to as the upstream oligonucleotide. The oligonucleotide that is in the most 3′ position, i.e., hybridized to the most 5′ position of the template, is referred to as the downstream oligonucleotide.

“dRpase,” as used herein, refers to an enzyme that excises a 5′ terminal AP endonucleotide.

“Nuclease” refers to an enzyme that cleaves nucleic acids at a phosphodiester linkeage or other linkage between nucleosides. Nucleases can be exonucleases, which remove one nucleotide at a time from the 3′ or 5′ end of a nucleic acid substrate, or endonucleases, which cleave a substrate nucleic acid at an internal linkage to produce two products with at least two nucleotides in each product.

Description.

One embodiment of the invention concerns labeling the 3′ portion of an upstream oligonucleotide with a recognition group, such as biotin. Generally, the recognition group attaches to the 3′ terminal nucleotide, but it can also attach to an internal nucleotide. The oligonucleotide is used in a polymerase or ligase reaction mixture, so it is extended by a polymerase, or ligated by a ligase to a downstream oligonucleotide. The 3′ terminal nucleotide of the upstream oligonucleotide can be blocked so that it cannot be extended or ligated unless the terminal nucleotide is removed. The block can be the recognition group itself, or can be another blocking group. Usually the recognition group is attached to the 3′ terminal nucleotide and also blocks the upstream oligonucleotide from being extended or ligated. The 3′ portion of the upstream oligonucleotide may also be non-complementary to the target sequence of the template nucleic acid to which the upstream oligonucleotide binds. A nuclease, such as the 3′-to-5′ proofreading exonuclease activity of certain polymerases, then removes the 3′ portion of the upstream oligonucleotide including the 3′ recognition group. If the 3′ terminal nucleotide is blocked, then at least that nucleotide must be removed before the polymerase can extend the upstream oligonucleotide, or before the ligase can ligate the upstream oligonucleotide to a downstream oligonucleotide. If the 3′ portion of the upstream oligonucleotide is non-complementary to the template, then the proofreading exonuclease will be more likely to remove it. Following removal of the 3′ portion of the upstream oligonucleotide, including the 3′ recognition group, the upstream oligonucleotide is extended by the polymerase or ligated to a downstream oligonucleotide by the ligase. Thus, the desired extended or ligated products lack the 3′ recognition group, while unreacted upstream oligonucleotides still contain the recognition group. By contacting the reaction mixture with a substrate that contains a group that binds the recognition group, the oligonucleotides with the recognition group can be removed from the desired products, which lack the recognition group. For instance, if the recognition group is biotin, the mixture can be contacted with a substrate containing avidin or streptavidin. This invention is applicable with all types of nucleic acid polymerase reaction mixes, including PCR, reverse transcriptase PCR, run-off analysis of RNA products, single base extension, and other assays. The invention is also applicable to ligase reactions, either alone or in combination with a polymerase reaction.

To remove unreacted oligonucleotides containing the recognition group, the reaction mixture is contacted with a substrate containing binding groups. The binding groups can be attached to a variety of supports, e.g., beads, microchannels, filters, or fibers such as agarose or cellulose. The separation between the bound oligonucleotides and the rest of the mixture can be accomplished in a variety of ways. Examples include gravitational settling of a solid substrate containing the binding group, centrifugation, magnetic separation (where the binding group is attached to a magnetic substrate), chromatography, filtration to remove a substrate containing the binding groups, filtration of the mixture through a filter containing binding groups, and electrophoresis. The substrate containing the binding group could be the binding group itself in a monomeric form. In this case, the binding group and the bound oligonucleotides could be separated from the mixture in a variety of ways, e.g., chromatography, electrophoresis, filtration, or aggregation and settling of the binding groups, as in the case of bivalent antibodies forming a cross-linked lattice with oligonucleotides comprising the antigen for the antibodies.

Another embodiment concerns labeling the 5′ portion of a downstream oligonucleotide with a recognition group, such as biotin. The recognition group can be attached to the 5′ terminal nucleotide or an internal nucleotide. The downstream oligonucleotide is used in a ligase reaction, to be ligated to an upstream oligonucleotide. The 5′ terminal nucleotide of the downstream oligonucleotide can be blocked so that it cannot be ligated unless the terminal nucleotide is removed. The block could be the recognition group itself, or could be another blocking group. Generally the 5′ recognition group is attached to the 5′ terminal nucleotide and blocks ligation of the downstream oligonucleotide to an upstream oligonucleotide until the 5′ recognition group is removed. The 5′ portion of the downstream oligonucleotide may be non-complementary to the target sequence of the template nucleic acid to which the oligonucleotide binds. In the reaction, a nuclease, such as a 5′-to-3′ exonuclease, removes the 5′ portion of the downstream oligonucleotide, including the 5′ recognition group. If the 5′ terminal nucleotide is blocked, then at least that nucleotide must be removed before the ligase can ligate the downstream oligonucleotide. The downstream oligonucleotide and the upstream oligonucleotide are designed so that following removal of the 5′ portion of the downstream oligonucleotide, including the 5′ recognition group, the free 5′-phosphate of the downstream oligonucleotide will lie adjacent to the 3′ hydroxyl of the upstream oligonucleotide. This allows the downstream oligonucleotide to be ligated efficiently by a ligase to the upstream oligonucleotide. Thus, the desired ligated product lacks the 5′ recognition group, while unreacted downstream oligonucleotide and some undesired products still contain the recognition group. By contacting the reaction mixture with a substrate that contains a group that binds the recognition group, the unreacted downstream oligonucleotide and undesired products containing the recognition group can be removed from the desired product, which lacks the recognition group. For instance, if the recognition group is biotin, the mixture can be contacted with a substrate containing avidin or streptavidin.

The advantages of some embodiments of the invention include easily removing unreacted oligonucleotides from a reaction mixture, thus achieving partial purification of the desired polynucleotide product. Removing the oligonucleotides is an important step, for instance, when an experimenter wishes to perform a second reaction on the polynucleotide product in which the oligonucleotides of the first reaction would interfere. This is the case, for instance, in nested PCR or sequencing PCR products.

Another advantage of some embodiments of the invention is that the nuclease digestion step of the invention can serve a proofreading function, increasing the yield of the desired product relative to the yield resulting from extension or ligation of oligonucleotide that has hybridized to non-target locations on the template. In polymerase chain reaction, this results, for instance, in reduced yield of primer dimers and other undesired reaction products.

Another advantage of some embodiments of the invention is that oligonucleotides that have been extended or ligated without prior removal of the recognition group are also removed from the reaction mixture. This improves the purity of the desired polynucleotide product by removing these undesired reaction products.

3′ Recognition-Group Method

Embodiments of the present invention include a method for removing unincorporated oligonucleotides from a reaction mixture. The method involves step (a), forming a mixture containing (i) a DNA polymerase or nucleic acid ligase, (ii) a nuclease, (iii) an upstream oligonucleotide having a 3′ portion and a 5′ portion, wherein the 3′ portion comprises a 3′ recognition group and a 3′ terminal nucleotide, and (iv) a template nucleic acid. The DNA polymerase or nucleic acid ligase and the nuclease can be the same or separate enzyme complexes. The method also involves step (b), digesting the 3′ portion of the upstream oligonucleotide with the nuclease, and step (c), extending the digested upstream oligonucleotide with the polymerase or ligating the digested upstream oligonucleotide to a downstream oligonucleotide with the ligase, wherein the extending or ligating forms a polynucleotide product. The method further involves step (d), contacting the mixture with a substrate comprising binding groups that bind the 3′ recognition group, to remove unincorporated upstream oligonucleotides from the reaction mixture. This method is hereinafter referred to as “the 3′-recognition-group method.”

In a specific embodiment of the 3′-recognition-group method, component (i) of the mixture is a nucleic acid polymerase, and step (c) is extending the upstream oligonucleotide with the polymerase to form the polynucleotide product. When the mixture comprises a nucleic acid polymerase, the mixture can contain two oligonucleotides. This will typically be the case when the mixture is a polymerase chain reaction mixture. Both oligonucleotides may contain the same 3′ recognition group or different 3′ recognition groups, or only one oligonucleotide may contain a 3′ recognition group.

In another embodiment of the 3′-recognition-group method, the reaction mixture is a reverse transcriptase-PCR reaction mixture.

In different embodiments of the 3′-recognition-group method, the DNA polymerase can be a DNA-directed DNA polymerase or a reverse transcriptase.

The polymerase and nuclease can be part of the same enzyme complex or be separate enzyme complexes.

In one embodiment where the polymerase is a reverse transcriptase, the mixture further contains a DNA-directed DNA polymerase. The reverse transcriptase and DNA-directed DNA polymerase can be the same or separate enzyme complexes. When they are the same enzyme complex, in specific embodiments the reverse transcriptase and DNA-directed DNA polymerase are, for instance, Anaerocellum thermophilum DNA polymerase, Bacillus pallidus DNA polymerase, Bacillus stearothermophilus DNA polymerase, Carboxydothermus hydrogenoformans DNA polymerase, Thermoactinomyces vulgaris DNA polymerase, Thermoanaerobacter thermohydrosulfuricus DNA polymerase, Thermosipho africanus DNA polymerase, Thermotoga neapolitana DNA polymerase, Thermus aquaticus DNA polymerase, Thermus thermophilus DNA polymerase, or Thermus Z05 DNA polymerase.

In one embodiment where the polymerase and nuclease are the same enzyme complex, the nuclease is a 3′-to-5′ exonuclease. In this embodiment, the enzyme complex can be, for instance, Pyrococcus furiosus polymerase THERMALACE, DEEP VENT DNA polymerase (Pyrococcus sp. GB-D), VENT DNA polymerase (Thermococcus litoralis), Bacillus stearothermophilus DNA polymerase, 9° Nₘ™ DNA polymerase (Thermococcus sp. strain 9° N-7), ACUPOL DNA polymerase, PROOFSTART DNA polymerase (Pyrococcus sp.), Pyrococcus woesei DNA polymerase, Thermococcus gorgonarius DNA polymerase, AMPLITHERM DNA polymerase, KOD DNA Polymerase (Pyrococcus kodakarensis), Thermococcus fumicolans DNA Polymerase, DYNAZYME EXT DNA polymerase (Thermus brockaianus), Thermosipho africanus DNA polymerase, Pyrodictium occultum DNA polymerase, Pyrococcus kodakarensis DNA polymerase, Thermotoga maritima DNA polymerase, Thermotoga neapolitana DNA polymerase, Bacillus pallidus DNA polymerase, Carboxydothermus hydrogenoformans DNA polymerase, Pyrococcus furiosus DNA polymerase, Pyrococcus sp. GB-D DNA polymerase, Thermococcus litoralis DNA polymerase, Thermococcus sp. strain 9° N-7 DNA polymerase, or Thermus brockaianus DNA polymerase.

In another specific embodiment of the invention, the polymerase and nuclease are separate enzyme complexes. In specific embodiments of this case, the polymerase is Thermus aquaticus DNA polymerase, Thermus thermophilus DNA polymerase, Z05 DNA polymerase (Thermus sp. Z05), SPS17 DNA polymerase (Thermus sp. SPS17), Thermoactinomyces vulgaris DNA polymerase, Thermoanaerobacter thermohydrosulfuricus DNA polymerase, Anaerocellum thermophilum DNA polymerase, or FY7 DNA polymerase (Thermoanaerobacter thermohydrosulfuricus FY7).

In another specific embodiment where the polymerase and nuclease are separate enzyme complexes, the nuclease is a mutant polymerase having 3′-to-5′ exonuclease activity that has lost its polymerase activity. The nuclease can be, for instance, a mutant of Pyrococcus furiosus polymerase THERMALACE, DEEP VENT DNA polymerase (Pyrococcus sp. GB-D), VENT DNA polymerase (Thermococcus litoralis), Bacillus stearothermophilus DNA polymerase, 9° Nₘ™ DNA polymerase (Thermococcus sp. strain 9° N-7), ACUPOL DNA polymerase, PROOFSTART DNA polymerase (Pyrococcus sp.), Pyrococcus woesei DNA polymerase, Thermococcus gorgonarius DNA polymerase, AMPLITHERM DNA polymerase, KOD DNA Polymerase (Pyrococcus kodakarensis), Thermococcus fumicolans DNA Polymerase, DYNAZYME EXT DNA polymerase (Thermus brockaianus), Thermosipho africanus DNA polymerase, Pyrodictium occultum DNA polymerase, Pyrococcus kodakarensis DNA polymerase, Thermotoga maritima DNA polymerase, Thermotoga neapolitana DNA polymerase, Bacillus pallidus DNA polymerase, Carboxydothermus hydrogenoformans DNA polymerase, Pyrococcus furiosus DNA polymerase, Pyrococcus sp. GB-D DNA polymerase, Thermococcus litoralis DNA polymerase, Thermococcus sp. strain 9° N-7 DNA polymerase, or Thermus brockaianus DNA polymerase.

In another specific embodiment of the invention where the polymerase and nuclease are separate enzyme complexes, the polymerase is a mutant form of a wild type polymerase having 3′-to-5′ exonuclease activity, where the mutant form has lost its exonuclease activity. The polymerase in this embodiment can be, for instance, mutant forms of Pyrococcus furiosus polymerase THERMALACE, DEEP VENT DNA polymerase (Pyrococcus sp. GB-D), VENT DNA polymerase (Thermococcus litoralis), Bacillus stearothermophilus DNA polymerase, 9° Nₘ™ DNA polymerase (Thermococcus sp. strain 9° N-7), ACUPOL DNA polymerase, PROOFSTART DNA polymerase (Pyrococcus sp.), Pyrococcus woesei DNA polymerase, Thermococcus gorgonarius DNA polymerase, AMPLITHERM DNA polymerase, KOD DNA Polymerase (Pyrococcus kodakarensis), Thermococcus fumicolans DNA Polymerase, DYNAZYME EXT DNA polymerase (Thermus brockaianus), Thermosipho africanus DNA polymerase, Pyrodictium occultum DNA polymerase, Pyrococcus kodakarensis DNA polymerase, Thermotoga maritima DNA polymerase, Thermotoga neapolitana DNA polymerase, Bacillus pallidus DNA polymerase, Carboxydothermus hydrogenoformans DNA polymerase, Pyrococcus furiosus DNA polymerase, Pyrococcus sp. GB-D DNA polymerase, Thermococcus litoralis DNA polymerase, Thermococcus sp. strain 9° N-7 DNA polymerase, or Thermus brockaianus DNA polymerase.

In one specific embodiment of the invention, the mixture contains a blend of two or more DNA polymerases having varying amounts of 3′-to-5′ exonuclease activity.

References for DNA polymerases useful in the invention are shown in the following tables.

DNA polymerases possessing 3′-to-5′
exonuclease activity.

3′>>5′

| | | |
|---|---|---|
| 9°Nₘ ™ DNA Polymerase | Yes | U.S. Pat. No. 5,756,334; |
| (Thermococcus sp. strain | | EP 0 701 000 |
| 9°N-7) | | |
| ACUPOL DNA Polymerase | Yes | |
| AMPLITHERM DNA | Yes | |
| polymerase | | |
| Bacillus pallidus DNA | Yes | |
| polymerase | | |
| Bacillus stearothermophilus | Yes | U.S. Pat. No. 5,834,253; |
| DNA polymerase | | U.S. Pat. No. 5,747,298; |
| | | U.S. Pat. No. 5,834,253; |
| | | EP 0 810 288; |
| | | EP 0 712 927 |
| Carboxydothermus | Yes | EP 0 834 569; |
| hydrogenoformans DNA | | WO 98/14589; |
| polymerase | | WO 98/14589 |
| DEEP VENT DNA | Yes | |
| Polymerase (Pyrococcus sp. | | |
| GB-D) | | |
| DYNAZYME EXT DNA | Yes | |
| polymerase (Thermus | | |
| brockaianus) | | |
| KOD DNA Polymerase | Yes | U.S. Pat. No. 6,008,025; |
| (Pyrococcus kodakarensis) | | EP 0 822 256 |
| PROOFSTART DNA | Yes | |
| polymerase (Pyrococcus | | |
| sp.) | | |
| Pyrococcus furiosus | Yes | U.S. Pat. No. 5,948,663; |
| polymerase THERMALACE | | U.S. Pat. No, 5,545,552; |
| | | U.S. Pat. No. 5,489,523; |
| | | WO 92/09689 |
| Pyrococcus kodakarensis | Yes | |
| DNA polymerase | | |
| Pyrococcus woesei DNA | Yes | |
| polymerase | | |
| Pyrodictium abyssi | Yes | U.S. Pat. No. 5,491,086 |
| Pyrodictium occultum DNA | Yes | U.S. Pat. No. 5,491,086 |
| polymerase | | |
| Thermococcus fumicolans | Yes | |
| DNA Polymerase | | |
| Thermococcus gorgonarius | Yes | EP 0 834 751; |
| DNA polymerase | | EP 0 834 570 |
| Thermosipho africanus DNA | Yes | WO 92/06202 |
| polymerase | | |
| Thermotoga maritima DNA | Yes | U.S. Pat. No. 5,420,029; |
| polymerase | | WO 97/09451 |
| Thermotoga neapolitana | Yes | U.S. Pat. No. 5,939,301; |
| DNA polymerase | | U.S. Pat. No. 6,077,664; |
| | | WO 96/10640; |
| | | WO 96/41014 |
| Thermococcus litoralis | Yes | U.S. Pat. No. 5,500,363; |
| | | U.S. Pat. No. 5,352,778; |
| | | U.S. Pat. No. 5,322,785; |
| | | U.S. Pat. No. 5,834,285; |
| | | U.S. Pat. No. 5,210,036; |
| | | 5,210,036; |
| | | EP 0 547 920 |
| VENT DNA Polymerase | Yes | |
| (Thermococcus litoralis) | | |

DNA polymerases lacking 3′>>5′
nuclease activity.

3′>>5′

| | | |
|---|---|---|
| Anaerocellum thermophilum | No | EP 0 835 935; |
| DNA polymerase | | WO 98/14588 |
| FY7 DNA polymerase | No | U.S. Pat. No. |
| (fragment of | | 5,744,312 |
| Thermoanaerobacter | | |
| thermohydrosulfuricus) | | |
| SPS17 DNA polymerase | No | |
| (Thermus sp. SPS17) | | |
| Taq DNA polymerase | No | |
| Thermoactinomyces vulgaris | No | |
| DNA polymerase | | |
| Thermoanaerobacter | No | U.S. Pat. No. |
| thermohydrosulfuricus DNA | | 5,744,312; |
| polymerase | | EP 0 866 868B1 |
| Thermus thermophilus DNA | No | |
| polymerase | | |
| Z05 DNA polymerase | No | |
| (Thermus sp. ZO5) | | |

Mutations to DNA polymerases removing 3′>>5′
exonuclease activity.

3′>>5′

| | | |
|---|---|---|
| Thermotoga maritima | No | U.S. Pat. No. 5,948,614; |
| | | WO 97/09451 |
| Thermotoga | No | U.S. Pat. No. 5,939,301; |
| neapolitana | | U.S. Pat. No. 6,077,664; |
| | | WO 96/10640; |
| | | WO 96/41014; |
| | | WO 97/09451 |
| Thermotoga | No | U.S. Pat. No. 5,500,363, |
| litoralis | | U.S. Pat. No. 5,756,334; |
| | | U.S. Pat. No. 5,352,778; |
| | | EP 0 547 920 |
| 9°Nₘ ™ DNA Poly- | No | U.S. Pat. No. 5,756,334 |
| merase | | |
| (Thermococcus sp. | | |
| strain 9°N-7) | | |
| Pyrococcus furiosus | No | U.S. Pat. No. 5,489,523 |
| Pyrococcus sp KOD | No | EP 0 822 256 |

DNA Polymerases with RT activity.

3′-5′

| | | |
|---|---|---|
| Anaerocellum thermophilum | Yes | WO 98/14588; |
| | | WO 98/14589; |
| | | WO 01/64954 |
| Bacillus paliidus DNA | | U.S. Pat. No. |
| polymerase | | 5,736,373 |
| Bacillus steraothermophilus | | WO 01/64954 |
| Carboxydothermus | Yes | EP 0 834 569; |
| hydrogenoformans | | WO 98/14589 |
| Thermoactinomyces vulgaris | Yes | WO 01/64838; |
| | | WO 01/64954 |
| Thermoanaerobacter | Yes | U.S. Pat. No. |
| thermohydrosulfuricus | | 5,744,312; |
| | | EP 09866 868 B1; |
| | | WO 97/21821; |
| | | WO 99/47539 |
| Thermosipho africanus | Yes | WO 92/06202 |
| Thermotoga neapolitana | Yes | U.S. Pat. No. |
| | | 5,912,155 |
| Thermus thermophilus | Yes | U.S. Pat. No. |
| | | 5,912,155 |
| Thermus ZO5 | Yes | |
| Thermus aquaticus | Yes | |

In a specific embodiment of the invention, the nuclease is inactive until an activation step is applied. This can be useful to prevent degradation of free oligonucleotides before they have hybridized to the template. In one embodiment of this invention, the nuclease is PROOFSTART DNA polymerase. Means of controlling the activation of the nuclease include chemical modification of the enzyme to inactivate it, where elevated temperature alters the chemical modification so as to activate the enzyme. See, e.g., U.S. Pat. Nos. 5,773,258 and 5,677,152. This can be accomplished by derivatizing the enzyme with a cyclic anhydride. The cyclic anhydride can be, for instance, succinic anhydride, citraconic anhydride, or cis-aconic anhydride. Another method of controlled inactivation is binding an antibody to the nuclease, where the antibody is inactivated by elevated temperatures. Another inactivation method is use of an aptamer or a peptide which binds to the nuclease at low temperatures and does not bind at elevated temperatures. Another inactivation method is partitioning the nuclease away from the oligonucleotide with a physical barrier. For instance, a wax barrier that melts at elevated temperatures can be used. An essential component required for enzyme activity, such as a divalent cation, can also be partitioned in the same way.

In another embodiment of the 3′-recognition-group method, the mixture includes a nucleic acid ligase, and the method includes the step of ligating the digested upstream oligonucleotide to a downstream oligonucleotide with the ligase to form the polynucleotide product. This embodiment can be used, for instance, in an oligonucleotide ligase assay. See Whiteley et al., U.S. Pat. No. 4,883,750 for a description of the oligonucleotide ligase assay.

In a specific embodiment of the 3′-recognition-group method, the 3′ terminal nucleotide of the upstream oligonucleotide is modified with a blocking group that prevents extension or ligation of the undigested upstream oligonucleotide.

In specific embodiments, the blocking group is a 3′-deoxynucleotide or a dideoxynucleotide. In other specific embodiments, the blocking group is 3′-phosphoglycoaldehyde, 3′-phosphate, 3′-mercapto, or 3′-amino. Phosphoglycoaldehyde refers to the group —OP(O)(OH)OCH₂CHO.

In a specific embodiment, the blocking group comprises the 3′ recognition group.

In one specific embodiment, the upstream oligonucleotide cannot be extended or ligated unless the 3′ recognition group is removed.

In one specific embodiment of the 3′-recognition-group method, the nuclease is a 3′-to-5′ exonuclease.

In one specific embodiment of the 3′-recognition-group method, the 3′ terminal nucleotide comprises all or part of the 3′ recognition group. In another specific embodiment, an internal nucleotide comprises all or part of the 3′ recognition group.

In one embodiment of the 3′-recognition-group method, the 3′ portion of the upstream oligonucleotide is non-complementary with the template. By “non-complementary” it is meant that the 3′ portion is not perfectly complementary in nucleotide sequence to the template. The 3′ portion can have a single base mismatch with the template, or can have no consecutive nucleotides complementary to the template, or can have a sequence of intermediate complementarity to the template. When the 3′ portion is non-complementary with the template, the 5′ portion of the upstream oligonucleotide will generally be more complementary to the template than the 3′ portion. The 5′ portion will generally be perfectly complementary to the template, but can have any sequence sufficiently complementary to the template that under the reaction conditions, the upstream oligonucleotide hybridizes to the template and can serve as a substrate for the polymerase to extend or the ligase to ligate to another hybridizing oligonucleotide.

Hybridization conditions are sequence dependent, and are different under different environmental parameters. Longer sequences hybridize specifically at higher temperatures. An extensive guide to the hybridization of nucleic acids is found in Tijssen, Laboratory Techniques in Biochemistry and Molecular Biology-Hybridization with Nucleic Acid Probes, page 1, chapter 2 “Overview of principles of hybridization and the strategy of nucleic acid probe assays” Elsevier, N.Y. (1993). Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences. For example, by “stringent conditions” or “stringent hybridization conditions” is intended conditions under which a probe will hybridize to its target sequence to a detectably greater degree than to other sequences (e.g., at least 2-fold over background). By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, an oligonucleotide probe is less than about 1000 nucleotides in length, preferably less than 500 nucleotides in length.

Typically, stringent conditions will be those in which the salt concentration involves less than about 1.5 M Na ion, typically about 0.01 to 1.0 M Na ion (or other cation), at pH 7.0 to 8.3 and a temperature of at least about 30° C. for short probes (e.g., 10 to 50 nucleotides) and at least about 60° C. for long probes (e.g., greater than 50 nucleotides).

The critical factors in specificity are the ionic strength and temperature of the reaction mixture. For DNA-DNA hybrids, the Tₘ can be approximated from the equation of Meinkoth and Wahl, Anal. Biochem. 138:267-284 (1984): Tₘ=81.5° C.+16.6 (log M)+0.41 (% GC)−0.61 (% form)−500/L; where M is the molarity of monovalent cations, % GC is the percentage of guanosine and cytidine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Alternatively, Tₘs can be determined from several commercially available programs such as PRIMER EXPRESS (Applied Biosystems). Tₘs can also be determined experimentally as described in Sambrook et al. (1989) Molecular Cloning: A Laboratory Manual (2d ed., Cold Spring Harbor Laboratory Press, Plainview, N.Y.).

Very highly stringent conditions are selected to be equal to, or slightly higher than, the Tₘ for a particular probe.

An example of stringent wash conditions is a 0.2×SSC wash at 65° C. for 15 minutes (see, Sambrook et al. (1989) Molecular Cloning: A Laboratory Manual (2d ed., Cold Spring Harbor Laboratory Press, Plainview, N.Y.) for a description of SSC buffer).

Tₘ is reduced by about 1° C. for each 1% of mismatching; thus, Tₘ, hybridization, and/or wash conditions can be adjusted to hybridize to sequences of the desired identity. For example, for a sequence with 90% identity, the Tₘ will be decreased approximately 10° C. Thus, if sequences with ≧90% identity are sought, the wash temperature will generally be about 10° C. lower than would be used to identify a perfectly complementary sequence.

Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 5, 6, 7, 8, 9, or 10° C. lower than the Tₘ; low stringency conditions can utilize a hybridization and/or wash at 11, 12, 13, 14, 15, or 20° C. lower than the Tₘ. Using these parameters, hybridization and wash compositions, and desired temperature, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology-Hybridization with Nucleic Acid Probes, Part 1, Chapter 2 (Elsevier, N.Y.); and Ausubel et al., eds. (1995) Current Protocols in Molecular Biology, Chapter 2 (Greene Publishing and Wiley-Interscience, New York). See also Sambrook et al. (1989) Molecular Cloning: A Laboratory Manual (2d ed., Cold Spring Harbor Laboratory Press, Plainview, N.Y.).

In one embodiment of the 3′-recognition-group method, all the nucleosides within the 3′ portion of the oligonucleotide are connected by linkages that are resistant to hydrolysis by the nuclease. In this case, if the nuclease cleaves the oligonucleotide, it will ordinarily cleave off the entire 3′ portion of the oligonucleotide. Linkages resistant to nucleases include methyl phosphonate linkages and phosphorothionate linkages.

In another embodiment of the 3′-recognition-group method, all the nucleosides within the 3′ portion of the upstream oligonucleotide are linked by phosphodiester linkages, and the 5′ portion of the upstream oligonucleotide comprises a linkage that is resistant to hydrolysis. For instance, the linkage resistant to hydrolysis could be a methyl phosphonate linkage or a phosphorothionate linkage. In this embodiment, a 3′-to-5′ exonuclease will tend to stop digestion at the linkage resistant to hydrolysis, leaving a 3′ terminal hydroxyl on the adjacent nucleotide. Thus, the linkage can be placed at the desired stop point for digestion.

The template nucleic acid in the method of the invention can be DNA or RNA.

In one embodiment of the invention, the substrate comprising the binding group is a size-exclusion-chromatography resin, and the mixture is passed through the resin. This method allows the removal of small molecules such as unreacted nucleotides at the same time that the unreacted oligonucleotides comprising the 3′ recognition group are removed. “Resin” as used here refers to both natural and synthetic polymers, such as dextran, polyacrylamide, agarose, etc., and mixtures thereof.

In one embodiment of the invention, the recognition group is a group recognized by an antibody, and the binding group is the antibody. For instance, the recognition group can be digoxygenin, fluorescein, or biotin, and the binding group can be an antibody that recognizes the appropriate recognition group.

When the recognition group is biotin, the binding group can also be, for example, avidin or streptavidin.

In another specific embodiment, the recognition group comprises phenylboronic acid, and the binding group comprises salicylhydroxamic acid. In another specific embodiment, the recognition group comprises salicylhydroxamic acid, and the binding group comprises phenylboronic acid.

In another specific embodiment, the recognition group is polyhistidine and the binding group is a nickel cation-chelate complex. Examples of chelators for the nickel cation are nitrilotriacetic acid or EDTA. The recognition group can also comprise a nickel cation-chelate, and the binding group be polyhistidine.

In another specific embodiment, the recognition group is a nucleotide sequence of the oligonucleotide, and the binding group is a complementary nucleotide sequence.

In another specific embodiment of the 3′-recognition-group method, the 3′ portion of the upstream oligonucleotide consists of L nucleotides, meaning nucleotides with L stereochemistry. L nucleic acids are generally not recognized by polymerases or ligases, so an upstream oligonucleotide whose 3′ portion consists of L nucleotides normally cannot be extended by a polymerase or ligated by a ligase unless the 3′ portion is removed. When the 3′ portion of the upstream oligonucleotide consists of L nucleotides, the binding group can be a complementary L oligonucleotide that hybridizes to the 3′ L nucleotides. Alternatively, other binding groups can be incorporated into the 3′ L nucleotide portion.

In another specific embodiment of the 3′-recognition-group method, the 3′ portion of the upstream oligonucleotide consists of peptide nucleic acid. When the 3′ portion of the upstream oligonucleotide consists of peptide nucleic acid, the binding moiety can be a complementary oligonucleotide that hybridizes to the 3′ peptide nucleic acid portion. Alternatively, other binding groups can be incorporated into the 3′ peptide nucleic acid portion.

In another embodiment of the 3′-recognition-group method, the upstream oligonucleotide comprises a modified nucleotide 5′ to the 3′ recognition group, and the nuclease cleaves the upstream oligonucleotide at the modified nucleotide. In one embodiment, the nuclease cleaves the upstream oligonucleotide at the modified nucleotide when the modified nucleotide is present in a duplex preferentially over when it is not in a duplex. In a specific embodiment where the nuclease cleaves at the modified nucleotide preferentially when it is in a duplex, the modified nucleotide is a ribonucleotide and the nuclease is RNAse H. In specific embodiments, the RNAse H is Thermus thermophilus DNA polymerase, Thermus thermophilus RNAse H, human RNAse H, or E. coli RNAse H.

Another modified nucleotide that can be used as a cleavage site is an abasic nucleotide. An abasic nucleotide residue can be generated by DNA glycosylases. DNA glycosylases are enzymes that remove bases in DNA through the hydrolysis of the N-glycosidic bond linking the base to its sugar. Most DNA glycosylases are highly selective for double-stranded DNA, with uracil glycosylase being an exception (Dodson M L, Michaels M L and Lloyd R S (1994) Unified Catalytic Mechanism for DNA Glycosylases. The Journal of Biological Chemistry 269 (52): 32709-32712.). The abasic site generated by a DNA glycosylase is referred to as an apurinic or apyrimidinic site, depending on whether the removed base was a purine or pyrimidine, respectively. Thus, they are called herein AP nucleotides, for apurinic or apyrimidinic. An AP nucleotide residue, such as would be generated by a DNA glycosylase, is shown in the middle molecule of the figure below.

DNA glycosylases can be divided into two groups. Monofunctional DNA glycosylases only catalyze the hydrolysis of the glycosidic bond, generating abasic sites. Bifunctional DNA glycosylases have an additional abasic site lyase activity, which results in cleavage of the 3′ C—O bond through _{CC}elimination. This is shown with the arrow to the right in the figure below. Some of the bifunctional enzymes also cleave the 5′ C—O bond through _{CC}elimination, yielding free 4-hydroxy-pent-2,4-dienal, and two DNA molecules terminating at free 5′-phosphoryl and 3′-phosphoryl termini at the nucleotides that flanked the AP nucleotide residue (Friedberg, E. C.; Walker, G. C., and Siede, W. 1995. DNA Repair and Mutagenesis. Washington, D.C.: ASM Press., page 156).

Oligonucleotides with an AP nucleotide residue can be cleaved 5′ to the AP nucleotide by apurinic/apyrimidinic endonucleases (AP endonucleases), as shown by the left arrow in the figure below. These leave a free 3′-OH and, on the AP nucleotide residue, a 5′-phosphate. AP endonucleases also cleave the 3′ terminal α,β-unsaturated aldehyde from the molecule in the top right in the figure below, leaving a 3′-OH terminus and free 4-hydroxy-5-phospho-2-pentenal.

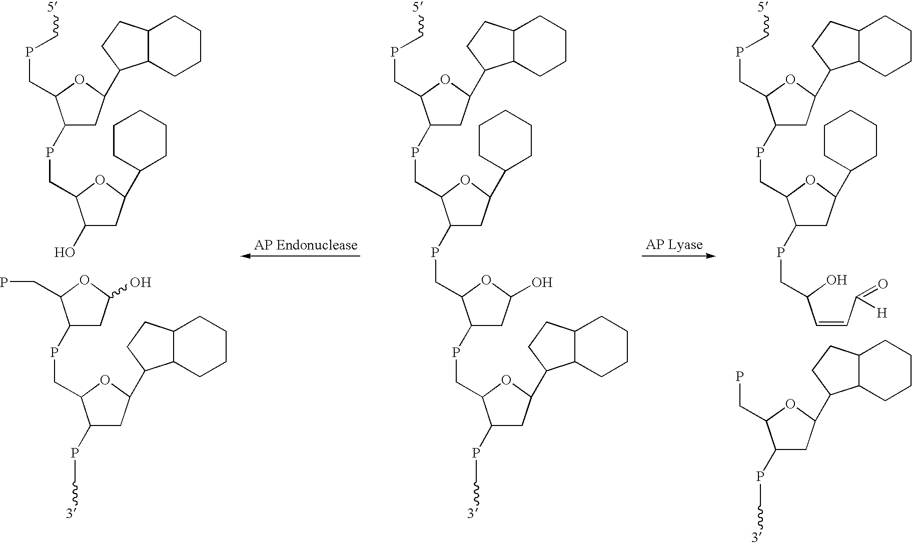

Two common AP endonucleases are exonuclease III, such as from E. coli, and APE 1 AP endonuclease. Another AP endonuclease is endonuclease IV from E. coli.

Exemplary DNA glycosylases and AP endonucleases and some details about their activities are shown in the tables below.

Monofunctional DNA Glycosylases (without lyase activity)

| | Substrate(s) | Preference | Result | Ref |
|---|---|---|---|---|
| Enzyme | | | | |
| 3-Methyladenine-DNA | 3-methyladenine, 3-ethyladenine, | Double-strand | Abasic | 3, 4 |
| glycosylase (eukaryotic) | 7-methylguanine, 7-ethylguanine, 3-methyl | | | |
| (ANPG) | guanine, 3-ethyl guanine, 1,N⁶-ethenoadenine, | | | |
| | Hypoxanthine, 8-oxoguanine, 3-alkylpurine | | | |
| 3-Methyladenine-DNA | 7-methylguanine, 3-methyladenine, | Double-strand | Abasic | 4, 6 |
| glycosylase II | O²-methylthymine, O²-methylcytosine, | | | |
| (Escherichia coli) (AlkA) | 5-formyluracil, 5-hydroxymethyluracil, | | | |
| | N²-3-ethenoguanine, 1,N⁶-ethenoadenine, | | | |
| | hypoxanthine, 7-alkylguanine, 7-alkylpurine, | | | |
| | 3-methyladenine, 3-methylguanine, | | | |
| | 7-methyladenine, N¹-carobxyethyladenine, | | | |
| | N⁷-carboxyethylguanine | | | |
| 3-Methyladenine-DNA | 3-methyladenine, 7-methylguanine, | Double-strand | Abasic | 2, 4, 6 |
| glycosylase I | 7-methyladenine, 3-methyladenine, | | | |
| (Escherichia coli) (tag) | O⁶-methylguanine, 3-ethyladeinine, | | | |
| | 3-methylguanine | | | |
| Mouse MPG | | | | |
| Thymine Mismatch-DNA | Thymine from G/T, C/T and T/T mismatches | Double-strand | Abasic | 5 |
| glycosylase | | | | |
| Lymphoblat Uracil DNA | Uracil | Double-strand | Abasic | 6 |
| glycosylase | | | | |
| Hypoxanthine DNA | Hypoxanthine | Double-strand | Abasic | 7 |
| N-glycosylase | | | | |

Bifunctional DNA Glycosylases (with lyase activity)

| Enzyme | Substrate | Preference | 3′ | 5′ | Ref |
|---|---|---|---|---|---|
| 8-Oxoguanine-DNA | 7,8-dihydro-8-oxoguanine, formamidopyrimidine, | Double-strand | Aldehyde | PO₄ | 8, 9, |
| glycosylase (OGG1) | 2,6-diamino-4-hydroxy-5-formamidopyrimidine, | | | | 10, 11 |
| | 8-oxoguanine | | | | |
| Endonuclease III (nth) | 5,6-dihydrothymine, 6-hydroxy-5,6-dihydrothymine, | Double-strand | Aldehyde | PO₄ | 12, 13 |
| Thymine glycol-DNA | cis-thymine glycol, trans-thymine glycol, | | | | |
| glycosylase | 5-hydroxy-5-methylhydantoin, methyltartonyl urea, | | | | |
| | urea, 5-hydroxycytosine, 5-hydroxyuracil, uracil | | | | |
| | glycol, dihydrouracil, 6-hydroxyuracil, glycol, β- | | | | |
| | ureidoisobutyric acid, 5-hydroxy-6-hydrothymine, | | | | |
| | 5,6-dihydrouracil, 5-hydroxy-6-hydrouracil, | | | | |
| | 5-hydroxy-2′-deoxycytidine, | | | | |
| | 5-hydroxy-2′-deoxyuridine | | | | |
| Endonuclease IV (nfo) | Urea, phosphoglycoaldehyde, phosphate, | | OH | PO₄ | 2, 13 |
| | deoxyribose-5-phosphate, and 4-hydroxy-2-pentenal | | | | |
| Endonuclease V | Pyrimidine dimer, inosine, deoxyuridine, | Double-strand | OH | PO₄ | 13, 14 |
| (Deoxyinosine | apurinic/apyrmidinic sites, urea, mismatches, | | | | |
| 3′-endonuclease) (nfi) | hairpins | | | | |
| Endonuclease VIII (nei) | 7,8-dihydro-8-oxoguanine, thymine glycol, | | PO₄ | PO₄ | 13 |
| | β-ureidoisobutyric acid, urea | | | | |
| Formamidopyrimidine | 8-oxo-7,8-dihydro-2′-deoxyguanosine, 7-methyl | Double-strand | PO₄ | PO₄ | 2, 9, |
| DNA glycosylase (Fpg) | guanine, | | | | 6, 15 |
| (mutM) | 2,6-diamino-4-hydroxy-5-N-methylformamidopyrimidine, | | | | |
| | 4,6-diamino-5-formamidopyrimidine, | | | | |
| | 5-hydroxy-2′-deoxycytidine, | | | | |
| | 5-hydroxy-2′-deoxyuridine, N⁷-methylguanine | | | | |
| MutY (micA) | 7,8-dihydro-8-oxoguanine, | | Aldehyde | PO₄ | 2, 16 |
| | 7,8-dihydro-8-oxo-adenine, A/C mismatch, A/G | | | | |
| | mismatch | | | | |
| K142A mutant of Mut Y | 7,8-dihydro-8-oxoguanine, | | PO₄ | PO₄ | 16 |
| | 7,8-dihydro-8-oxo-adenine, A/C mismatch, A/G | | | | |
| | mismatch | | | | |
| Thymine hydrate DNA | | Double-strand | | | 6 |
| glycosylase | | | | | |
| (Escherichia coli) | | | | | |
| Pyrimidine dimer DNA | | Double-strand | | | 6 |
| Glycosylase (M. luteus) | | | | | |
| 8-Hydroxyquanine | 8-hydroxyguanine, | Double-strand | PO₄ | PO₄ | 17, 18 |
| endonuclease | 8-oxo-7,8-dihydro-2′-deoxyguanosine | | | | |
| Yeast Endonuclease | Imidazole-ring fragmented formamidopyrimidine | | Aldehyde | PO₄ | 19, 20 |
| three-like glycosylase | Formamidopyrimidine-containing; 8-oxoguanine; | | | | |
| (NTG1) (yOgg2) | thymine glycol, N7-metghylated formamidopyrimidine | | | | |

Apurinic/apyrimidinic (AP)-endonucleases

| Enzyme | Substrate | Preference | 3′ | 5′ | Ref |
|---|---|---|---|---|---|
| Endonuclease IV | Abasic sites | Double-strand | OH | PO₄ | 2 |
| APE 1 AP | Abasic sites | Double-strand | OH | PO₄ | 21, 22 |
| endonuclease | | | | | |
| Exonuclease III | Abasic site | Double-strand | OH | PO₄ | 2 |
| Endonuclease IV | Abasic site | | | | 11 |
| (nfo) | | | | | |

Endonuclease IV and exonuclease III can remove phosphoglycoaldehyde, phosphate, deoxyribose-5-phosphate and 4-hydroxy-2-pentenal residues from the 3′ terminus of duplex DNA (2).
Exonuclease III also has 3′-phosphatase activity (2).
APE 1 AP endonuclease has 3′-phosphatase and 3′-phosphodiesterase activity. APE 1 AP endonuclease, endonuclease IV, and exonuclease III can remove the 3′-phospho-α,β-unsaturated aldehyde terminus produced by the β-elimination reaction produced by a lyase reaction (11).

Nucleases sometimes leave a 3′ terminal phosphate, which can prevent extension or ligation of the upstream oligonucleotide. Thus, it is sometimes necessary to include a 3′ phosphatase in the mixture to remove this 3′ terminal phosphate. In specific embodiments, the mixture further contains a 3′ phosphatase. In specific embodiments, the 3′ phosphatase is exonuclease III, exonuclease IV, or yeast AP endonuclease.

In one specific embodiment of the method involving cleavage at a modified nucleotide preferentially in a duplex, the modified nucleotide comprises 8-oxo-7,8-dihydro-2′-deoxyguanosine; 7-methylguanine; 2,6-diamino4-hydroxy-5-N-methylformamidopyrimidine; 4,6-diamino-5-formamidopyrimidine; 5-hydroxy-2′-deoxycytidine; 5-hydroxy-2′-deoxyuridine; or N⁷-methylguanine; and the nuclease is formamido-pyrimidine-DNA glycosylase, and the mixture further contains a 3′ phosphatase.

In another specific embodiment of the method involving cleavage at a modified nucleotide preferentially in a duplex, the modified nucleotide contains 7,8-dihydro-8-oxoguanine; formamidopyrimidine; 2,6-diamino-4-hydroxy-5-formamidopyrimidine; or 8-oxoguanine; and the nuclease is 8-oxoguanine DNA glycosylase, and the mixture further contains an AP endonuclease.

In another specific embodiment of the method involving cleavage at a modified nucleotide preferentially in a duplex, the modified nucleotide contains 5,6-dihydrothymine; 6-hydroxy-5,6-dihydrothymine; cis-thymine glycol; trans-thymine glycol; 5-hydroxy-5-methylhydantoin; methyltartonyl urea; urea; 5-hydroxycytosine; 5-hydroxyuracil; uracil glycol; dihydrouracil; 6-hydroxyuracil; glycol; β-ureidoisobutyric acid; 5-hydroxy-6-hydrothymine; 5,6-dihydrouracil; 5-hydroxy-6-hydrouracil; 5-hydroxy-2′-deoxycytidine; or 5-hydroxy-2′-deoxyuridine; and the nuclease is endonuclease III or thymine glycol-DNA glycosylase, and the mixture further contains an AP endonuclease.

In another specific embodiment, the modified nucleotide is an AP nucleotide and the nuclease is an AP endonuclease. In specific embodiments when the modified nucleotide is an AP nucleotide, the nuclease is exonuclease III, endonuclease IV, APE 1 AP endonuclease, or yeast AP endonuclease.

In a specific embodiment of the 3′-recognition-group method, the 5′ portion of the upstream oligonucleotide contains a 5′ recognition group that is different from the 3′ recognition group. After the reaction, the desired product will contain the 5′ recognition group but not the 3′ recognition group, while the upstream oligonucleotide will contain both recognition groups. Thus, contacting the reaction mixture with a substrate containing binding groups that bind the 3′ recognition group removes undigested upstream oligonucleotides. If the mixture is then contacted with a substrate containing binding groups that bind the 5′ recognition group, the desired product can be removed from the reaction mixture. Thus, another specific embodiment of the invention is the method wherein the 5′ portion of the upstream oligonucleotide contains a 5′ recognition group that is different from the 3′ recognition group. In this embodiment, the method can further involve (after contacting the mixture with a substrate containing binding groups that bind the 3′ recognition group) the step of contacting the mixture with a substrate containing binding groups that bind the 5′ recognition group.

5′-Recognition-Group Method

The present invention provides another method for removing unincorporated oligonucleotides from a reaction mixture. The method involves step (a), forming a mixture containing (i) a nucleic acid ligase, (ii) a nuclease, (iii) a downstream oligonucleotide having a 3′ portion and a 5′ portion, wherein the 5′ portion comprises a 5′ recognition group and a 5′ terminal nucleotide, and (iv) a template nucleic acid. The ligase and nuclease can be the same or separate enzyme complexes. The method also involves step (b), digesting the 5′ portion of the downstream oligonucleotide with the nuclease; and step (c), ligating the digested downstream oligonucleotide to an upstream oligonucleotide with the ligase, wherein the ligating forms a polynucleotide product. The method further involves step (d), contacting the mixture with a substrate containing binding groups that bind the 5′ recognition group, to remove unincorporated downstream oligonucleotides from the reaction mixture. This method is hereinafter referred to as “the 5′-recognition-group method.”

In a specific embodiment of the 5′-recognition-group method, the 5′ terminal nucleotide of the downstream oligonucleotide is modified with a blocking group that prevents ligation of the undigested downstream oligonucleotide. In particular embodiments, the blocking group is 5′-mercapto, 5′-amino, 5′-diphosphate, 5′-triphosphate, or a 5′-deoxynucleotide.

In a specific embodiment, the blocking group contains the 5′ recognition group. In one specific embodiment, the downstream oligonucleotide cannot be ligated unless the 5′ recognition group is removed.

In one specific embodiment of the 5′-recognition-group method, the nuclease is a 5′-to-3′ exonuclease. In one specific embodiment, the 5′-to-3′ exonuclease preferentially digests single stranded DNA. One such exonuclease is Rec J_{f}, available from New England Biolabs.

In another specific embodiment of the 5′-recognition-group method, the nuclease is inactive until an activation step is applied.

In one specific embodiment of the 5′-recognition-group method, the 5′ terminal nucleotide contains all or part of the 5′ recognition group. In another specific embodiment, an internal nucleotide contains all or part of the 5′ recognition group.

In one embodiment of the 5′-recognition-group method, the 5′ portion of the downstream oligonucleotide is non-complementary with the template. By “non-complementary” it is meant that the 5′ portion is not perfectly complementary in nucleotide sequence to the template. The 5′ portion can have a single base mismatch with the template, or can have no consecutive nucleotides complementary to the template, or can have a sequence of intermediate complementarity to the template. When the 5′ portion is not complementary to the template, the 3′ portion of the downstream oligonucleotide will generally be more complementary to the template than the 5′ portion. The 3′ portion will generally be perfectly complementary to the template, but can have any sequence sufficiently complementary to the template that under the reaction conditions, the downstream oligonucleotide hybridizes to the template and can serve as a substrate for the ligase to ligate to another hybridizing oligonucleotide.

In one embodiment of the 5′-recognition-group method, all the nucleosides within the 5′ portion of the downstream oligonucleotide are connected by linkages that are resistant to hydrolysis by the nuclease. In this case, if the nuclease cleaves the downstream oligonucleotide, it will ordinarily cleave off the entire 5′ portion of the downstream oligonucleotide. Linkages resistant to nucleases include methyl phosphonate linkages and phosphorothionate linkages.

In another embodiment of the 5′-recognition-group method, all the nucleosides within the 5′ portion of the downstream oligonucleotide are linked by phosphodiester linkages, and the 3′ portion of the downstream oligonucleotide comprises a linkage that is resistant to hydrolysis. For instance, the linkage resistant to hydrolysis, could be a methyl phosphonate linkage or a phosphorothionate linkage. In this embodiment, a 5′-to-3′ exonuclease will tend to stop digestion at the linkage resistant to hydrolysis, leaving a 5′ terminal phosphate on the adjacent nucleotide. Thus, the linkage can be placed at the desired stop point for digestion.

In one embodiment of the 5′-recognition group method, the 5′ portion of the downstream oligonucleotide consists of L nucleotides.

The template nucleic acid in the methods of the invention can be DNA or RNA.

In one embodiment of the 5′-recognition-group method, the substrate containing the binding group is a size-exclusion-chromatography resin, and the mixture is passed through the resin. This method allows the removal of small molecules such as unreacted nucleotides at the same time that the unreacted oligonucleotides comprising the 5′ recognition group are removed. “Resin” as used here refers to both natural and synthetic polymers, such as dextran, polyacrylamide, agarose, etc., and mixtures thereof.

In another specific embodiment of the 5′-recognition-group method, the 5′ portion of the downstream oligonucleotide consists of L nucleotides, meaning nucleotides with L stereochemistry. L nucleic acids are generally not recognized by ligases, so a downstream oligonucleotide whose 5′ portion consists of L nucleotides normally cannot be ligated by a ligase at its 5′ end unless the 5′ portion is removed. In another specific embodiment of the 5′-recognition-group method, the 5′ portion of the downstream oligonucleotide is peptide nucleic acid.

In another embodiment of the 5′-recognition-group method, the downstream oligonucleotide contains a modified nucleotide 3′ to the 5′ recognition group, wherein the nuclease cleaves the downstream oligonucleotide at the modified nucleotide. In one embodiment, the nuclease cleaves at the modified nucleotide when the modified nucleotide is present in a duplex preferentially over when it is not in a duplex.

In one embodiment where the nuclease preferentially cleaves at the modified nucleotide when the modified nucleotide is present in a duplex, the modified nucleotide is a ribonucleotide and the nuclease is RNAse H.

In one specific embodiment of the 5′ recognition group method, where the nuclease preferentially cleaves at the modified nucleotide when the modified nucleotide is in a duplex, the modified nucleotide is or contains 8-oxo-7,8-dihydro-2′-deoxyguanosine; 7-methylguanine; 2,6-diamino-4-hydroxy-5-N-methylformamidopyrimidine; 4,6-diamino-5-formamidopyrimidine; 5-hydroxy-2′-deoxycytidine; 5-hydroxy-2′-deoxyuridine; or N⁷-methylguanine; and the nuclease is formamido-pyrimidine-DNA glycosylase.

In another specific embodiment of the 5′-recognition-group method, where the nuclease preferentially cleaves at the modified nucleotide when it is in a duplex, the modified nucleotide contains 8-hydroxyguanine, and the nuclease is 8-hydroxyguanine endonuclease or N-methylpurine DNA glycosylase.

In another specific embodiment of the 5′-recognition-group method, where the nuclease preferentially cleaves at the modified nucleotide when it is in a duplex, the modified nucleotide contains 7,8-dihydro-8-oxoguanine; formamidopyrimidine; 2,6-diamino-4-hydroxy-5-formamidopyrimidine; or 8-oxoguanine; and the nuclease is 8-oxoguanine-DNA glycosylase.

In another specific embodiment of the 5′-recognition-group method, where the nuclease preferentially cleaves at the modified nucleotide when it is in a duplex, the modified nucleotide is an AP nucleotide. In a specific embodiment when the modified nucleotide is an AP nucleotide, the nuclease is a DNA glycosylase with lyase activity.

Nucleases sometimes leave a free 5′-OH, which cannot be a substrate for ligation. Thus, it is sometimes necessary to include a 5′ kinase in the mixture to add one phosphate to the 5′-OH. In specific embodiments, the mixture further contains a 5′ kinase.

In a specific embodiment of the 5′ recognition-group method, the downstream oligonucleotide contains a modified nucleotide 3′ to the 5′ recognition group, the nuclease cleaves the downstream olgonucleotide at the modified nucleotide and leaves a 5′ terminal AP nucleotide, and the mixture further contains a deoxyribophosphodiesterase (dRpase). “dRpase” is defined herein as an enzyme that excises a 5′ terminal AP endonucleotide. An example is the E. coli Rec J protein (Friedberg, E. C.; Walker, G. C., and Siede, W. 1995. DNA Repair and Mutagenesis. Washington, D.C.: ASM Press.).

In a specific embodiment of the 5′-recognition-group method, the 3′ portion of the downstream oligonucleotide contains a 3′ recognition group that is different from the 5′ recognition group. After the reaction, the desired product will contain the 3′ recognition group but not the 5′ recognition group, while the undigested downstream oligonucleotide will contain both recognition groups. Thus, contacting the reaction mixture with a substrate containing binding groups that bind the 5′ recognition group removes undigested downstream oligonucleotides. If the mixture is then contacted with a substrate containing binding groups that bind the 3′ recognition group, the desired product is removed from the reaction mixture. Thus, another specific embodiment of the 5′-recognition-group method is the method wherein the 3′ portion of the downstream oligonucleotide contains a 3′ recognition group that is different from the 3′ recognition group. In this embodiment, the method can further involve (after contacting the mixture with a substrate comprising binding groups that bind the 5′ recognition group) the step of contacting the mixture with a substrate containing binding groups that bind the 3′ recognition group.

Recognition/Binding Groups

Recognition groups can be attached to nucleotides or oligonucleotides, or incorporated into oligonucleotides, at any synthetically feasible position by techniques known in the art. Binding groups can also be attached to supports at any synthetically feasible position. For example, suitable reagents and reaction conditions are disclosed, e.g, in Advanced Organic Chemistry, Part B: Reactions and Synthesis, Second Edition, Cary and Sundberg (1983); Advanced Organic Chemistry, Reactions, Mechanisms, and Structure, Second Edition, March (1977); Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999). Labeling reagents and prelabeled nucleotides are also available from commercial suppliers, such as Applied Biosystems Corp, Foster City, Calif.; Glen Research Corp., Sterling, Va.; and Prolinx, Inc., Redmond, Wash. Recognition groups, or recognition-group-labeled nucleotides, can be incorporated into oligonucleotides by oligonucleotide synthesizers as one of the nucleotide units incorporated into the oligonucleotide.

Supports with attached binding groups are available from many commercial suppliers. For instance, streptavidin-coated magnetic beads are available from Dynal, Oslo, Norway.

Supports incorporating salicylhydroxamic acid and phenylboronic acid groups are available from Prolinx, Redmond, Wash. Nickel-NTA complex-coated magnetic agarose beads are available from Qiagen.

Suitable recognition group-labeled nucleotides include 6-FAM™-dU (Applied Biosystems) and Biotin-dU, as shown below.

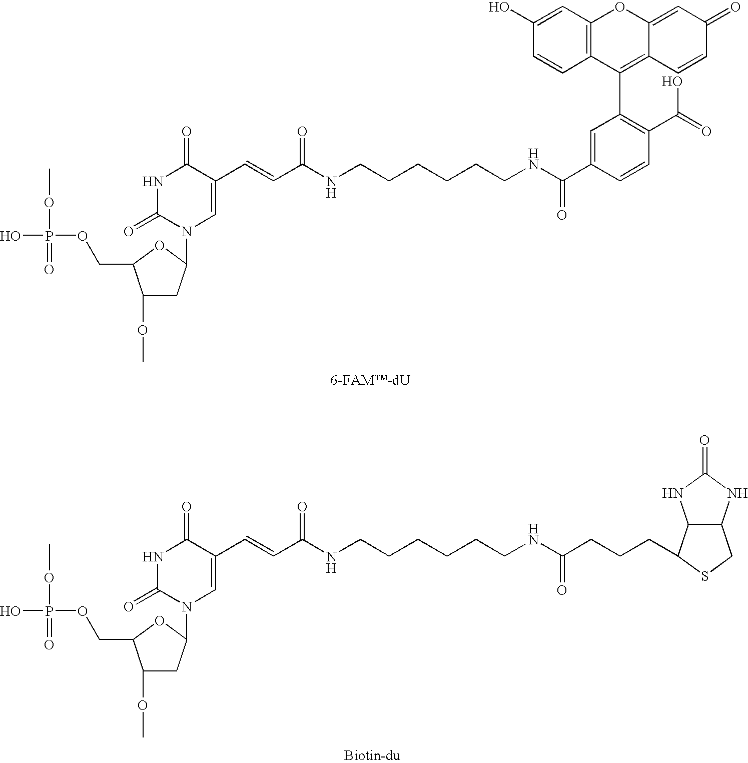

Another suitable recognition group ready to attach to nucleotides is N-hydroxysuccinimide-tetramethyl rhodamine (NHS-TAMRA) (Applied Biosystems). N-hydroxysuccinimide esterified recognition groups will react to form attachments with amino groups. Thus, the TAMRA-NHS ester can react with 3′ amino oligonucleotides to attach a recognition group to the 3′ terminal nucleotide of the oligonucleotide.

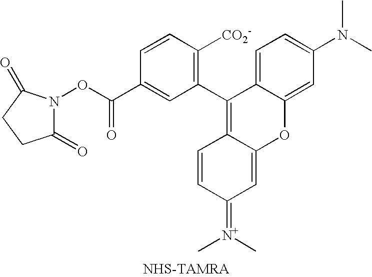

In order that the invention may be more readily understood, reference is made to the following examples which are intended to illustrate the invention, but not limit the scope thereof.

### EXAMPLE 1

PCR reactions of 100 μl were carried out in 0.2 ml MICROAMP tubes with PCR buffer (Applied Biosystems), 200 μM each dNTP, 0.25 μM each primer, 2.5 units enzyme, and 25 ng phage lambda DNA. The reactions were heated to 95° C. for 10 minutes, then thermal cycled for 30 cycles of 94° C. for 15 seconds and 68° C. for 1 minute, the last cycle being followed by an extension of 72° C. for 7 minutes and a final hold at 4° C. Reactions were performed with a non-proofreading enzyme (AMPLITAQ, Applied Biosystems) or a proofreading polymerase (PFU TURBO, Stratagene). All the reactions used TAMRA-PC02 as the reverse primer. The forward primer for Mismatch #1 was F-PC01-BdT, where the two 3′ terminal nucleotides, biotin-dT and C, are mismatched. The forward primer for Mismatch #2 was PC01 -FAM, where the two 3′ terminal nucleotides C and 3′-fluorescein dT CPG, are mismatched. The reverse primer for Match #1 was F-PC01. Primer sequences are shown below, with the 3′ end on the right.

TAMRA-PC02
12GGTTATCGAAATCAGCCACAGCGCC

where 1=NHS-TAMRA (Applied Biosystems), and 2=amino link (Applied Biosystems)

F-PC01-BdT
1GATGAGTTCGTGTCCGTACAACT2C

where 1=6-FAM (Applied Biosystems), and 2=biotin-dT (Glen Research)

F-PC01
1GATGAGTTCGTGTCCGTACAACT

where 1=6-FAM (Applied Biosystems)

PC01-FAM
GATGAGTTCGTGTCCGTACAACTC1

where 1=3′-fluorescein-dT CPG (Glen Research)

The expected product was 500 bp. Gel electrophoresis revealed that the non-proofreading enzyme was able to generate the expected product with the matched primer, but not with either mismatched primer. The proofreading enzyme, in contrast, produced the expected product in good yield with both mismatched and matched reverse primers. (Data not shown.)

### EXAMPLE 2

PCR reactions of 100 μl were carried out in 0.2 ml MICROAMP tubes with PCR buffer at 2 mM MgSO₄, 200 μM each dNTP, 0.25 μM each primer, 2.5 units PFU TURBO polymerase, and 25 ng lambda DNA. The thermal cycle program was as in Example 1. One μl of the product reaction mix was analyzed using an ABI 310 Genetic Analyzer (Applied Biosystems) using the run module GS STR POP4 (C), 1 sec injection, 7.5 kV/injection, 15 kV/run, 60° C. for 30 minutes.

A portion of the product reaction mixture was contacted with magnetic streptavidin-coated beads (Dynal, Oslo, Norway) to remove the unincorporated biotinylated primer. Samples of the reaction mixture before and after contact with the streptavidin-coated beads were analyzed by electrophoresis and fluorescent detection. These experiments showed that the beads removed unincorporated biotinylated primer, without removing the unincorporated TAMRA-labeled primer or the product, which has TAMRA and FAM labels but no biotin.

### REFERENCES

1. Dodson M L, Michaels M L and Lloyd R S (1994). Unified Catalytic Mechanism for DNA Glycosylases. The Journal of Biological Chemistry 269 (52): 32709-32712.
2. Friedberg, E. C.; Walker, G. C., and Siede, W. (1995). DNA Repair and Mutagenesis. Washington, D.C.: ASM Press.
3. Helland D E, Male R, Haukanes B I, Olson L, Haugan I, and Kleppe K. (1987). Properties and Mechanism of Action Of Eukaryotic 3-Methyladenine-DNA Glycosylases. J. Cell. Sci. Suppl. 6: 139-146.
4. Schärer O D, Nash H M, Jiricny J, Laval J, and Verdine G L. (1998). Specific Binding of a Designed Pyrrolidine Abasic Site Analog to Multiple DNA Glycosylases. The Journal of Biological Chemistry 273(15): 8592-8597.
5. Neddermann P., and Jiricny J. (1993). The Purification of a Mismatch-Specific Thymine-DNA Glycosylase from HeLa Cells. The Journal of Biological Chemistry. 268(28): 21218-21224.
6. Duncan, B K. (1981). DNA Glycosylases, p. 565-586. In P.D. Poyer (ed), The Enzymes, Vol. XIV. Academic Press, Inc., New York.
7. Karran P, and Lindahl T (1978). Enzymatic excision of free hypoxanthine from polydeoxynucleotides and DNA containing deoxyinosine monophosphate residues. The Journal of Biological Chemistry 253(17): 5777-5879.
8. Ishchenko A A, Bulychev N V, Maksakova G A, Johnson F, and Nevinsky G A (1997). Recognition and Conversion of Single-Stranded Oligonucleotide Substrates by 8-Oxoguanine-DNA Glycosylase from Escherichia coli. Biochemistry (Moscow) 62 (2): 204-211.
9. Alamo M J P, Jurado J, Francastel E, and Laval F. (1998). Rat 7,8-Dihydro-8-oxoguanine DNA Glycosylase: Substrate Specificity, Kinetics and Cleavage Mechanism At An Apurinic Site. Nucleic Acids Research 26(22): 5199-5202.
10. Asagoshi K, Yamada T, Terato H, Ohyama Y, Monden Y, Arai T, Nishimura S, Aburatani H, Lindahl T, Ide H. (2000). Distinct Repair Activities of Human 7,8-dihydro-8-oxoguanine DNA Glycosylase and Formamidopyrimidine DNA Glycosylase for Formamidopyrimidine and 7,-8-Dihydro-8-oxoguanine. The Journal of Biological Chemistry 275(7): 4956-4964.
11. Hill J W, Hazra T K, Izumi T, and Mitra S. (2001). Stimulation of Human 8-Oxoguanine-DNA Glycosylase By AP-Endonuclease: Potential Coordination Of The Initial Steps In Base Excision Repair. Nucleic Acids Research. 29(2): 430-438.
12. Okano K, and Kambara H. (1995). DNA Probe Assay Based On Exonuclease III digestion Of Probes Hybridized On Target DNA. Analytical Biochemistry 228: 101-108.
13. Jiang D, Hatahet Z, Melamede R J, Kow Y W, and Wallace S S. (1997). Characterization of Escherichia coli Endonuclease VIII. The Journal of Biological Chemistry 272 (51): 32230-32239.
14. Yao M, and Kow Y W. (1997). Further Characterization of Escherichia coli Endonuclease V: Mechanism of Recognition For Deoxyinosine, Deoxyuridine, and Base Mismatches in DNA. The Journal of Biological Chemistry. 272(49): 30774-30779.
15. Hatahet Z, Kow Y W, Purmal A A, Cunningham R P, and Wallace S S. (1994). New Substrates For Old Enzymes: 5′-Hydroxy-2′-Deoxycytidine And 5-Hydroxy-2′-Deoxyuridine Are Substrate For Escherichia coil Endonuclease III and Formamidopyrimidine DNA-Glycosylase While 5′-Hydroxy-2′-Deoxyuridine Is A Substrate For Uracil DNA-Glycosylase. The Journal of Biological Chemistry 269(29): 18814-18820.
16. Wright P M, Yu J, Cillo J, Lu A-L. (1999). The Active Site Of The Escherichia coil MutY DNA Adenine Glycosylase. The Journal of Biological Chemistry. 274(41): 29011-29018.
17. Chung, M H, Kasai H, Jones D S, Inoue H, Ishikawa H, Ohtsuka E, Nishimura S (1991). An endonuclease activity of Eschericia coli that specifically removes 8-hydroxygunine residues from DNA. Mutat Res 254(1): 1-12.
18. Tchou J, Kasai H, Shibutani S, Chung M-H, Laval J, Grollman A P, and Nishimura S (1991). 8-Oxoguanine (8-hydroxyguanine) DNA Glycosylase And Its Substrate Specificity. Proc. Natl. Acad. Sci. USA 88: 4690-4694.
19. Alseth I, Eide L, Pirovano M, Rognes T, Seeberg E, and Bjør\dot{a}s (1999). The Saccharomyces cerevisiae Homologues of Endonuclease III from Escherichia coli, Ntg1 and Ntg2, Are Both Required for Efficient Repair of Spontaneous and Induced Oxidative DNA Damage in Yeast. Molecular and Cellular Biology. 19(5): 3779-3787.
20. Bruner S D, Nash H M, Lane W S, and Verdine G L (1998). Repair Of Oxidatively Damaged Guanine in Saccharomyces cerevisiae By An Alternative Pathway. Current Biology. 8:393-403.
21. Chou K-M, Kukhanova M, and Cheng Y-C. (2000). A Novel Action of Human Apurinic/Apyrimidinic Endonuclease: Excision of L-Configuation Deoxyribonucleoside Analogs From The 3′ Termini of DNA. The Journal Of Biological Chemistry. 275(40):31009-31015.
22. Wilson III D M, Takeshiita M, Grollman A P, Demple B. (1995). Incision Acitivity of Human Apurinic Endonuclease (Ape) At Abasic Site Analogs in DNA. The Journal of Biological Chemistry. 270(27): 16002-16007.

All references cited herein are hereby incorporated by reference.

1-62. (canceled)

63. A method for removing unincorporated oligonucleotides from a reaction mixture, the method comprising:
(a) forming a mixture comprising:
(i) a nucleic acid ligase;
(ii) a nuclease;
(iii) a downstream oligonucleotide having a 3′ portion and a 5′ portion, wherein the 5′ portion comprises a 5′ recognition group and a 5′ terminal nucleotide; and
(iv) a template nucleic acid;
wherein (i) and (ii) are the same or separate enzyme complexes;
(b) digesting the 5′ portion of the downstream oligonucleotide with the nuclease;
(c) ligating the digested downstream oligonucleotide to an upstream oligonucleotide with the ligase, wherein the ligating forms a polynucleotide product; and
(d) contacting the mixture with a substrate comprising binding groups that bind the 5′ recognition group, to remove unincorporated downstream oligonucleotides from the reaction mixture.

64. The method of claim 63, wherein the 5′ terminal nucleotide of the downstream oligonucleotide is modified with a blocking group that prevents ligation of the undigested downstream oligonucleotide.

65. The method of claim 64, wherein the blocking group is 5′-mercapto, 5′-amino, 5′-diphosphate, 5′-triphosphate, or a 5′-deoxynucleotide.

66. The method of claim 64, wherein the blocking group comprises the 5′ recognition group.

67. The method of claim 63, wherein the downstream oligonucleotide cannot be ligated unless the 5′ recognition group is removed.

68. The method of claim 63, wherein the nuclease is a 5′-to-3′ exonuclease.

69. The method of claim 68, wherein the nuclease is Rec J_{f}.

70. The method of claim 63, wherein the nuclease is inactive until an activation step is applied.

71. The method of claim 63, wherein the 5′ terminal nucleotide comprises all or part of the 5′ recognition group.

72. The method of claim 63, wherein an internal nucleotide of the downstream oligonucleotide comprises all or part of the 5′ recognition group.

73. The method of claim 63, wherein the 5′ portion of the downstream oligonucleotide is non-complementary with the template.

74. The method of claim 63, wherein all the nucleosides within the 5′ portion of the downstream oligonucleotide are linked by linkages that are resistant to hydrolysis by the nuclease.

75. The method of claim 74, wherein the linkages are methyl phosphonate linkages.

76. The method of claim 74, wherein the linkages are phosphorothionate linkages.

77. The method of claim 63, wherein all the nucleosides within the 5′ portion of the downstream oligonucleotide are linked by phosphodiester linkages, and the 3′ portion of the downstream oligonucleotide comprises a linkage that is resistant to hydrolysis.

78. The method of claim 77, wherein the linkage resistant to hydrolysis is a methyl phosphonate linkage or a phosphorothionate linkage.

79. The method of claim 63, wherein the 5′ portion of the downstream oligonucleotide consists of L nucleotides.

80. The method of claim 63, wherein the template nucleic acid is DNA.

81. The method of claim 63, wherein the template nucleic acid is RNA.

82. The method of claim 63, wherein the substrate is a size-exclusion-chromatography resin.

83. The method of claim 63, wherein the recognition group is a group recognized by an antibody, and the binding group is the antibody.

84. The method of claim 83, wherein the recognition group is digoxygenin.

85. The method of claim 83, wherein the recognition group is fluorescein.

86. The method of claim 83, wherein the recognition group is biotin.

87. The method of claim 63, wherein the recognition group is biotin and the binding group is avidin or streptavidin.

88. The method of claim 63, wherein the recognition group comprises phenylboronic acid and the binding group comprises salicylhydroxamic acid.

89. The method of claim 63, wherein the recognition group comprises salicylhydroxamic acid and the binding group comprises phenylboronic acid.

90. The method of claim 63, wherein the recognition group is polyhistidine and the binding group is a nickel cation-chelate complex.

91. The method of claim 63, wherein the recognition group is a nucleotide sequence of the downstream oligonucleotide and the binding group is a complementary nucleotide sequence.

92. The method of claim 63, wherein the downstream oligonucleotide comprises a modified nucleotide 3′ to the 5′ recognition group, and wherein the nuclease cleaves the downstream oligonucleotide at the modified nucleotide.

93. The method of claim 92, wherein the nuclease cleaves the downstream oligonucleotide at the modified nucleotide when the modified nucleotide is present in a duplex preferentially over when it is not in a duplex.

94. The method of claim 93, wherein the modified nucleotide is a ribonucleotide and the nuclease is an RNAse H.

95. The method of claim 94, wherein the RNAse H is Thermus thermophilus DNA polymerase, Thermus thermophilus RNAse H, human RNAse H, or E. coli RNAse H.

96. The method of claim 93, wherein the modified nucleotide comprises 8-oxo-7,8-dihydro-2′-deoxyguanosine; 7-methylguanine; 2,6-diamino-4-hydroxy-5-N-methylformamidopyrimidine; 4,6-diamino-5-formamidopyrimidine; 5-hydroxy-2′-deoxycytidine; 5-hydroxy-2′-deoxyuridine; or N⁷-methylguanine; and the nuclease is formamido-pyrimidine-DNA glycosylase.

97. The method of claim 93, wherein the modified nucleotide comprises 8-hydroxyguanine, and the nuclease is 8-hydroxyguanine endonuclease or N-methylpurine DNA glycosylase.

98. The method of claim 93, wherein the modified nucleotide comprises 7,8-dihydro-8-oxoguanine; formamidopyrimidine; 2,6-diamino-4-hydroxy-5-formamidopyrimidine; or 8-oxoguanine; and the nuclease is 8-oxoguanine-DNA glycosylase.

99. The method of claim 93, wherein the modified nucleotide is an AP nucleotide and the nuclease is a DNA glycosylase with lyase activity.

100. The method of claim 92, wherein after digesting, the nuclease leaves a 5′ terminal AP nucleotide, and the mixture further comprises a dRpase.

101. The method of claim 63, wherein the 3′ portion of the downstream oligonucleotide comprises a 3′ recognition group that is different from the 5′ recognition group.

102. The method of claim 101, further comprising after step (d), step (e): contacting the mixture with a substrate comprising binding groups that bind the 3′ recognition group.


SEQUENCE LISTING (4 sequences)

SEQ ID NO: 1 - DNA, 25 bp, Phage SV 40
ggttatcgaaatcagccacagcgcc

SEQ ID NO: 2 - DNA, 25 bp, Phage SV 40
gatgagttcgtgtccgtacaacttc

SEQ ID NO: 3 - DNA, 23 bp, Phage SV 40
gatgagttcgtgtccgtacaact

SEQ ID NO: 4 - DNA, 25 bp, Phage SV 40
gatgagttcgtgtccgtacaactct